(19) United States
(12) Patent Application Publication
(10) Pub. No.: US 2026/0207678 A1
(43) Pub. Date: Jul. 23, 2026

(54) FREEZE DRIED EXOSOME-BASED COMPOSITION AND METHOD OF PREPARATION THEREOF

(71) Applicant: TECH CELL INNOVATIONS PVT. LTD., Delhi (IN)

(72) Inventor: Suchi GUPTA, Delhi (IN)

(21) Appl. No.: 19/157,349

(22) PCT Filed: Feb. 15, 2024
(86) PCT No.: PCT/IN2024/050152

§ 371 Date: Aug. 16, 2025

(30) Foreign Application Priority Data
Feb. 16, 2023 (IN) 202211046455

(51) Int. Cl. A61K 35/50; A61K 9/19; A61K 9/50; A61P 19/02

(52) U.S. Cl. CPC A61K 35/50; A61K 9/19; A61K 9/5068; A61P 19/02

(57) ABSTRACT

The present disclosure provides a composition comprising a combination of stem cell derived exosome and amniotic membrane in freeze dried form for regeneration of skin and bone/cartilage cells. The present disclosure also provides a simple and cost-effective method for preparing said composition for wound healing and tissue regeneration from biomedical wastes including placenta and umbilical cord.

### FIELD OF THE DISCLOSURE

The present disclosure relates to freeze dried bio-vesicle based composition and its method of preparation. More particularly, the present disclosure provides a composition comprising a combination of exosome and amniotic membrane in freeze-dried form for skin and bone-cartilage tissue regeneration.

### BACKGROUND ART OF THE DISCLOSURE

Mounting evidence supports stem cells as a promising way to heal human tissue. There are different components of stem cells that aid in repair and regeneration, of which exosomes, a class of tiny double membrane bound vesicles also known as extracellular vesicles (EVs) have attracted the attention of the scientists and clinicians worldwide. Their small size and off-the-shelf applications offer clinicians a safer and simpler alternative to cellular therapy. Moreover, several pre-clinical studies reported till date have strengthened the available data on the safety and efficacy of these exosomes. Over the past years, various market competitors have emerged in the field of exosome therapeutics and diagnostics.

As per a Market Research Report, the global exosome research products market is projected to reach USD 661 million by 2026 from USD 144 million in 2021, at a CAGR of 35.6% during the forecast period. The growth of this market is driven by factors such as the increasing funding for life sciences research, the high global prevalence of cancer, and the increasing interest in exosome-based procedures. However, most of the exosome-based products are being used for dermal applications and other cosmetic purposes.

Despite these advancements, exosome-based developments including products, their safety and efficacy studies are still naïve. Furthermore, exosome-based therapies also face various challenges such as challenging task of isolation, purification, and large-scale production of exosomes represents a direct obstacle to the development of exosome-based treatment from bench to bed (Riau et al., 2019).

Also, there are ethical concerns involved with the source and preparation of such compositions. Thus, there is a need for development of improved and effective therapeutic exosomal products with potential applications for promoting skin and tissue regeneration and treatment of damaged cells and tissues. Addressing the need, the present disclosure explores and harnesses this potential of the exosomes in combination with amniotic membrane to build up the tissue matrix and thereby promoting tissue regeneration and wound healing.

### OBJECTIVES OF THE DISCLOSURE

The primary objective of the present disclosure is to provide a composition comprising freeze dried biovesicles such as exosomes and amniotic membrane.

Another object of the present disclosure is to provide a simple and cost-effective method for preparing a composition for wound healing from biomedical wastes including placenta and umbilical cord.

Another object of the present disclosure is to provide a composition for wound healing.

Another object of the present disclosure is to provide a composition for bone-cartilage regeneration and treatment of osteoarthritis.

Still another object of the present disclosure is to provide a kit comprising the composition of the present disclosure along with an instruction manual.

Additional objects, advantages, and novel features of the disclosure will be set forth in part in the description which follows, and in part will become apparent to those skilled in the art upon examination of the following or may be learned by practice of the disclosure.

### SUMMARY OF THE DISCLOSURE

The following summary is provided to facilitate an understanding of some of the innovative features unique to the disclosed embodiments and is not intended to the full description of the disclosure. A full appreciation of the various aspects of the preferred embodiments disclosed herein can be gained by taking the entire specification, claims, drawings, and abstract as a whole.

In an aspect of the present disclosure, a novel value-added composition is provided comprising wharton's jelly stem cell derived exosomes and amniotic membrane from biological wastes such as umbilical cord or placenta for enhancing the angiogenesis and/or vascularization, more particularly, healing of skin wounds and cartilage defects.

In an important embodiment, the present disclosure provides a safe and easy to use freeze dried formulation comprising exosome and amniotic membrane.

In another aspect of the present disclosure, a process is provided for the preparation of the composition of the present disclosure involving isolation of mesenchymal stem cells and their characterization, isolation of extracellular vesicles and exosomes, freeze drying of the extracellular vesicles and exosomes, separation of amniotic membrane, lyophilization and optional solubilization followed by mixing the lyophilized exosome powder and amniotic membrane powder in a therapeutically effective amount.

In an embodiment, the composition of the present disclosure is in the form of a nano-formulation comprising freeze dried exosome powder and amniotic membrane powder.

In another aspect of the present disclosure, a nano-formulation comprising bioactive combination of exosomes and amniotic membrane is provided for tissue regeneration.

### BRIEF DESCRIPTION OF THE DRAWINGS

The drawings described herein are for illustrative purposes only of selected embodiments and not all possible implementations and are not intended to limit the scope of the present disclosure. The disclosure itself, however, both as to organization and method of operation, may best be understood by reference to the detailed description which follows taken in conjunction with the accompanying drawings in which:

FIG. 1 provides a schematic illustration for the development of the exosome-based EXO-AM composition of the present disclosure, according to an embodiment of the present disclosure.

FIG. 2 shows the lyophilized powder exosomes and its characterization by electron microscopy for cup shaped morphology and size distribution profile done by nanoparticle tracking analysis, according to an embodiment of the present disclosure.

FIG. 3 shows the tube formation assay using exosomes pre and post lyophilization, according to an embodiment of the present disclosure.

FIG. 4 is a representative image showing (a) powdered form of AM after denudation as confirmed by (b) H&E staining. The AM powdered form was sterilized by (c) Gamma sterilization (15 kGy) and (d) 1.5% agarose gel showing absence of DNA in AM after decellularization as compared to native AM.

FIG. 5 is a representative image showing lyophilized exosome powder at two different concentrations (50 μg & 25 μg). Size distribution analysis shows size <200 nm; cup shaped morphology was maintained as evaluated by Electron Microscopy and expression of exosome specific protein was maintained in different batches.

FIG. 6 shows a cell migration assay using HaCaT cells to evaluate the functionality of Amniotic Membrane (AM) and Exosomes. Representative brightfield phase contrast images at 0 h and 24 h time points for cell migration analysis (Magnification 4× & scale bar 50 μm). Bar graph representing the percentage area closure in the treated groups. The asterisks (*) indicate significant differences amongst the group, with * p<0.05, ** p<0.01, *** p<0.001 and *** p<0.0001.

FIG. 7 shows a tube formation assay using HUVEC cells to evaluate the functionality of Amniotic Membrane (AM) and Exosomes. (a) Representative brightfield phase contrast images at 0 h and 6 h time points for tube formation analysis (Magnification 4× & scale bar 50 μm). Bar graph representing the (b) Total tube length, (c) Branch counts and (d) Loop Count in the treated groups. The asterisks (*) indicate significant differences amongst the group, with * p<0.05, ** p<0.01, *** p<0.001 and *** p<0.0001.

FIG. 8 shows a collagen content assay for native and powdered form of Amniotic Membrane (AM). Bar graph representing the total collagen concentration. The asterisks (*) indicate significant differences compared amongst each group, with * p<0.05.

FIG. 9 shows a cytokine concentration in different preparations of human amniotic membrane (native vs powder) stored at different temperatures for different time points. (a) Concentration of Hyaluronic Acid (ng/mg) & (b) Concentration of HGF, EGF, IL-10, TGF-β3 and VEGF (pg/mg). The asterisks (*) indicate significant differences amongst the group, with * p<0.05, ** p<0.01, *** p<0.001 and *** p<0.0001.

FIG. 10 shows the details of the wound contraction study. Representative images showing gross view and quantification of wound area in all the groups on days 3, 7 and 12 post-wounding.

FIG. 11 shows representative images showing hematoxylin & eosin staining of the wound sections in untreated vs treated group on day 12. The red arrows indicate newly formed blood vessels, orange arrows indicate inflammatory cells such as neutrophils, yellow arrows indicate fibroblast and asterisk (*) indicates collagenization.

FIG. 12 shows representative images showing haematoxylin & eosin staining of the wound sections in all the groups including healthy control, untreated (sham group) and treated groups on day 12 at different magnifications—Group1: Healthy Group 2—Untreated; Group 3—2.5 mg AM; Group 4—1.25 mg AM; Group 5—50 μg Exo; Group 6—Combination A (50 μg Exo: 2.5 mg AM) & Group 7—Combination B (50 μg Exo: 1.25 mg AM).

FIG. 13 provides a schematic illustration for the development of the exosome-based EXO-AM composition of the present disclosure, according to an embodiment of the present disclosure.

FIG. 14 is a representative figure showing, gross image of rat knee of both healthy and OA group. The H& E image showing proliferation of spindle shaped cells and multilayered structure in treated group as compared to untreated/sham (Magnification; 200×).

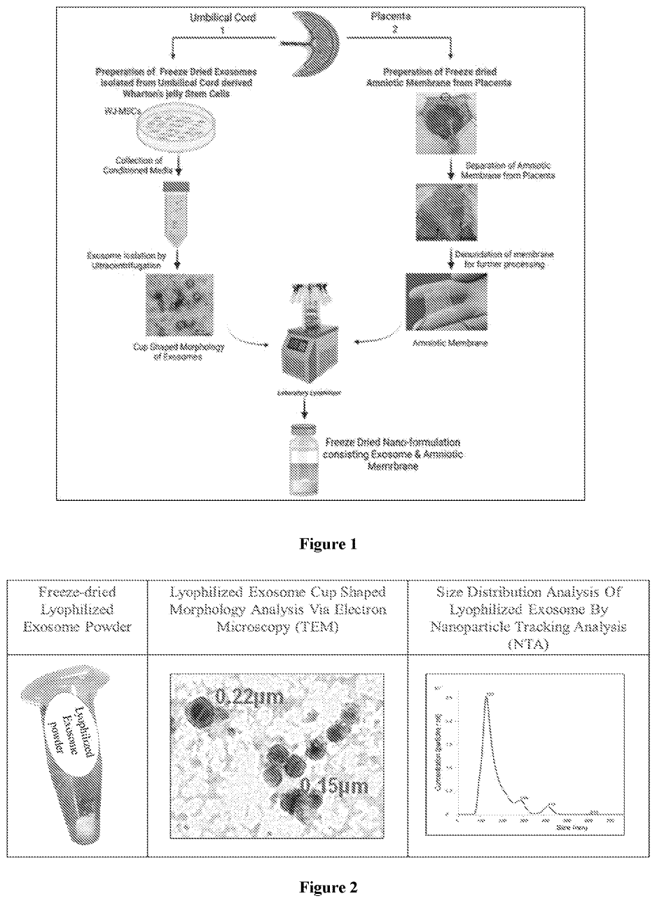

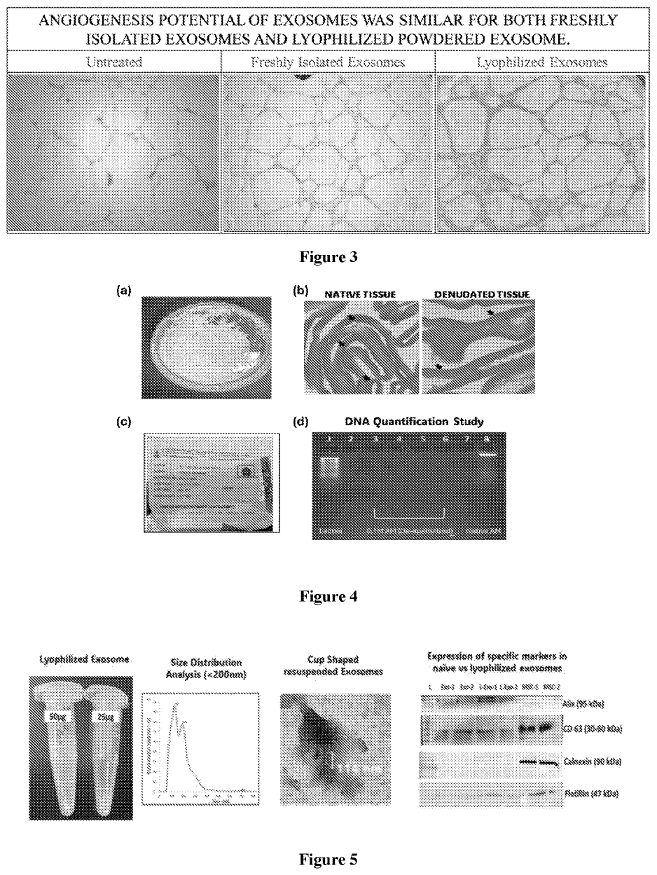

### DETAILED DESCRIPTION OF THE DISCLOSURE

The details of one or more embodiments of the disclosure are set forth in the accompanying description below including specific details of the best mode contemplated by the inventors for carrying out the disclosure, by way of example. It will be apparent to one skilled in the art that the present disclosure may be practiced without limitation to these specific details.

### Abbreviations Used

MSC—Mesenchymal Stem Cell
AM—Amniotic membrane
EXO—Exosomes
CM—Conditioned Media
OA—Osteoarthritis

The use of “comprise”, “comprises”, “comprising”, “contain”, “contains”, “containing”, “include”, “includes”, and “including” are not intended to be limiting. It is to be understood that both the foregoing general description and this detailed description are exemplary and explanatory only and are not restrictive.

Unless otherwise defined, scientific and technical terms used herein shall have the meanings that are commonly understood by those of ordinary skill in the art. Further, unless otherwise required by context, singular terms shall include pluralities and plural terms shall include the singular.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

A novel exosome-based composition is provided comprising exosomes (EXO) and amniotic membrane (AM). Particularly, the composition comprises exosomes from mesenchymal stem cells and placenta derived amniotic membrane. The present disclosure provides an economical approach for preparation of exosome-based composition as it involves the use of biomedical waste such as wharton's jelly for isolation of exosomes and amniotic membrane from discarded placental tissue.

The composition of the present disclosure is in the form of a nano-formulation comprising freeze dried exosome powder and amniotic membrane powder in a therapeutically effective amount.

In an embodiment, the composition of the present invention comprises exosomes and amniotic membrane in a therapeutically effective amount.

In some embodiments, the composition of the present invention comprises exosome and amniotic membrane in a therapeutically effective ratio range of 1:10 to 1:50.

In some embodiments of the present invention, the exosomes are derived from wharton's jelly stem cells.

In some embodiments of the present invention, the amniotic membrane is derived from placental tissue.

In some embodiments of the present invention, the exosome and amniotic membrane are freeze dried and are in a powdered form.

In some embodiments of the present invention, the exosome powder and amniotic membrane powder is preferably present in a ratio of about 1:25:1:50.

In some embodiments of the present invention, the composition is a freeze-dried nano-formulation.

In some embodiments of the present invention, the composition is in the form of a powder, spray, gel, ointment, liquid.

In some embodiments, the composition of the present invention can be administered topically, intraarticularly, intravenously, subcutaneously at the site of injury.

In some embodiments, a method of preparing the composition of the present invention is provided comprising the steps of:
a) lyophilizing exosomes at −50° C. for 24 hrs to obtain exosome powder;
b) optionally solubilizing amniotic membrane and then lyophilizing at −50° C. for 24 hrs to obtain amniotic membrane powder;
c) sterilizing the powders obtained in step a) and b); and
d) mixing the sterilized exosome and amniotic membrane powder obtained in step c) in a ratio range of 1:10 to 1:50 to obtain the final composition.

In some embodiments of the present invention, the amniotic membrane is solubilized by an enzymatic method.

In some embodiments, the present invention provides a kit comprising the composition of the present invention along with an instruction manual.

In some embodiments, the composition of the present invention can be used for healing wounds or treatment of bone degenerative diseases selected from but not limited to osteoarthritis, osteoporosis, osteopenia, osteonecrosis.

In some embodiments, the composition of the present invention is in the form of a powder, gel, spray when used for wound healing.

In some embodiments, the composition of the present invention is in the form of a powder which can be later solubilized to be used as an intra-articular injection for treatment of bone-cartilage degenerative disorders.

In some embodiments, the present invention provides a method of treating wound by applying the composition as claimed in claim 1.

In some embodiments, the present invention provides a method of treating bone degenerative diseases by administering the composition as claimed in claim 1 intraarticularly, intravenously, subcutaneously at the site of injury.

In an embodiment, the composition of the present invention comprises 0.025 mg to 0.2 mg of exosomes and 1 mg to 12.5 mg amniotic membrane.

In an exemplary embodiment, the therapeutically effective ratio of exosomes and amniotic membrane in 1:25-1:50.

In another embodiment of the present disclosure, the exosomes are isolated from Mesenchymal Stem Cells derived from umbilical cord and amniotic membrane is derived from placental tissue.

Further areas of applicability of the present disclosure will become apparent from the detailed description provided hereinafter. It should be understood that the detailed description and specific examples while indicating the preferred embodiment of the disclosure, are intended for purposes of illustration only and are not intended to limit the scope of the disclosure.

### EXAMPLES

### Example 1: Preparation of Freeze-Dried Amniotic Membrane and Exosome Formulation

### 1A. Preparation of the Lyophilized Amniotic Membrane (AM Powder)

A total of 10 placentas were processed for the separation of the amniotic membrane and the formation of powder. Placentas was obtained from the donors undergoing caesarean surgery for delivery in AIIMS, New Delhi. The prior consent was taken from all the donors and all serological parameters were evaluated as per DBT-ICMR guidelines (HIV, HBV, HCV test). The processed amniotic membrane was then lyophilized at −50° C.

Following lyophilization, a sterility check was performed for any possible bacterial and fungal contaminants at the Department of Microbiology, AIIMS. Gamma irradiation (15 kGy) was performed as an additional sterilization measure.

The sterilized amniotic membrane was then de-epithelialized to promote better cell proliferation and differentiation, better structural integrity, as well as more uniform cell outgrowth by treating with 0.1M NaOH for a period of 5-10 minutes. Post de-epithelization, about 50 ng/mg of DNA was isolated with an approximate yield of 500 mg of powdered amniotic membrane (FIG. 4).

### 1B. Preparation of Lyophilized Exosomes Powder

Total 10 umbilical cords were obtained from donors undergoing caesarean surgery for delivery in AIIMS, New Delhi. The prior consent was taken from the donors and all serological parameters were evaluated as per DBT-ICMR guidelines (HIV, HBV, HCV test) were processed for isolation of Mesenchymal Stem Cells (MSCs) by the explant method. Standard explant culture method was used where the cord was brought under sterile conditions to the laboratory and then given two washes with 2% antibiotic solution for 30 minutes at room temperature. The cords were cut open to expose the jelly like tissue (wharton's jelly). This jelly like tissue was scraped off using blade and then chopped into small pieces. These pieces were placed on each well of 6 well plate (4-5 pieces per well) and then drop wise 100 μl of MSC specific culture media was added. The plate was kept in the incubator and then next day 2 ml of media was added.

10-20 vials of 1 million cells/ml of cryomix from each donor MSCs (a total of 100 vials) were taken from cryopreservation. The revival efficiency of the vials was 80-90%.

The MSCs were cultured in serum free media (Gibco, Thermo Fisher, USA) for isolation of exosomes. A pooling strategy was performed to prevent donor to donor variation. Pooling strategy was performed where the MSC obtained from 5 donors after complete characterization and sterility parameters were mixed in 1:1 ratio and then cultured and expanded. These pooled MSC population were re-characterized for surface marker profiling, PDT and tri-lineage differentiation to ensure, they maintain their characteristics. These MSCs were cultured and then CM was collected from them for exosome isolation.

This was followed by ultracentrifugation to isolate the exosomes. The extract was filtered by a 0.22 μm filter to remove any debris/larger particles.

The filtrate was lyophilized at −50° C. A sterility test was performed to check for any contaminants whether the sample was negative for Mycoplasma, bacterial and fungal strains. The approximate yield obtained was 300-500 μg protein equivalent exosomes per 250 ml CM per 30 million cells (P2-P5).

Nanoparticle tracking analysis (NTA) was done to check the size distribution of the exosomes which were determined to be at least less than 200 nm. The TEM for morphology indicated them to be Cup shaped. This was followed by Western Blotting for exosome specific marker expression (CD63, Alix & Flotillin; negative marker: Calnexin) (FIG. 5).

### 1C. Preparation of Solubilized Amniotic Membrane

For osteoarthritis application, before preparation of the amniotic membrane powder, the amniotic membrane was solubilized by an enzymatic method.

### Preparation of the AM Prior to Solubilization

A total of 10 placentas were obtained from patients undergoing an elective caesarean section and willing for a repeat serological test after 3 months. The donor blood serology was negative for HIV, Hepatitis B and C and VDRL at baseline. The placentas were kept in a sterile bottle containing transport solution (Normal saline, Heparin (5000 IU), Penicillin 50 μg/ml, Streptomycin 50 μl/ml, Gentamycin in 200 μg/ml &Amphotericin-B 2.5 μg/ml). The placentas were transferred into a sterile tray under laminar flow hood and the amnion was separated from the chorion by blunt dissection. The amniotic membrane (AM) was washed thoroughly with a washing solution (Normal saline, Penicillin 50 μg/ml, Streptomycin 50 μl/ml, Gentamycin in 200 μg/ml &Amphotericin-B 2.5 μg/ml) to remove the blood clots. After removing all the blood clots, the AM was placed on the tray with the stromal surface up. The epithelial layer was removed by treating with 0.1M NaOH for 5-10 minutes and then thoroughly washed with Normal saline.

### Solubilization of Amniotic Membrane

The pepsin was weighed in the ratio of 1:10 (for 100 mg of AMG add 10 mg of pepsin) and added to 10 ml of 0.01 N HCl. The solution was mixed well and then the pepsin solution was carefully added to the AM. The AM was incubated on the rocker at 37° C. for overnight incubation (24 hrs). Next day, the AM was completely solubilized. The solution was centrifuged at 3000 g for 10 minutes at 4° C. to pellet down any leftover particles and the supernatant was collected. The supernatant was freeze dried immediately and then lyophilized to form fine powder. The powder was then weighed as per the required composition and aliquoted.

The AM post solubilization was resuspended in milliq and then added to 50 μg exosome solution in the ratio range of 1:25 to 1:50. The entire mixture was then freeze dried and lyophilized to obtain the final combination for osteoarthritis studies.

### Example 2: Preparation of the Formulation for Functional Assessment

### 2A. Powdered Amniotic Membrane

For functional studies of the powdered amniotic membrane (AM), Migration Assay, Total collagen content assay and cytokine estimation were performed. The conditioned media (CM) collected from the native and powdered form of AM were used. The CM was prepared by adding 1 ml of Serum Free Media (STEMPRO® MSC SFM CTS, Thermo Fisher Scientific) to 5×5 cm² native and equivalent amount of powdered form of AM. The samples were then placed in a CO₂ incubator at 37° C. for 24 hours to allow for proper conditioning. After the incubation period, the CM was collected and aliquoted in sterile Eppendorf tubes. To preserve its integrity, the CM aliquots were snap-frozen using liquid nitrogen and stored at −80° C. until further use.

### 2B. Lyophilized Exosomes

Migration Assay and Angiogenesis assay using in-vitro cell-based methods was performed for functional studies of lyophilized exosomes. The lyophilized exosomes (50 μg) were resuspended in 1×PBS (sterile) and compared with non-lyophilized Fresh exosomes (50 μg).

### 2C. Combination-Based Formulation

For functional studies of combination-based formulation, 50 μg lyophilized Exosomes and 5 mg AM powder (lyophilized or optionally solubilized) were mixed together and stored for 24 h at 4° C. 1 ml of 1×PBS was added on the next day and then samples were placed in a CO₂ incubator at 37° C. for 24 hours to allow for proper conditioning. After the incubation period, the CM was collected and aliquoted in sterile Eppendorf tubes. To preserve its integrity, the CM aliquots were snap-frozen using liquid nitrogen and stored at −80° C. until further use.

In order to avoid batch to batch variations, different batches of AM and exosomes were pooled and aliquoted.

### Example 3: Functional Assays-Methodology and Results

### 3A. Migration Assay

The assay was conducted using the scratch assay technique combined with mitomycin C treatment. The goal of this assay was to investigate the migratory potential of cells under controlled conditions. The scratch assay involved creating a straight scratch or wound on a confluent cell monolayer using a sterile pipette tip. Following this, mitomycin C, a cell proliferation inhibitor, was applied to the cells to prevent cell division and ensure that the observed cell migration is primarily due to cell movement rather than cell proliferation. The migration of cells into the scratched area was monitored and analyzed over a specified period of time, providing valuable insights into cell migration dynamics and potential factors influencing the process. This cell migration assay using the scratch technique and mitomycin C treatment offers a powerful tool for studying cellular motility and the underlying mechanisms involved, contributing to our understanding of cell migration processes in various physiological and pathological contexts.

a. Methodology

Human keratinocyte cell lines (HaCaT) (obtained from National Repository at National Centre for Cell Science, Pune, Maharashtra, India) were utilized, and the cells were cultured in DMEM-F12 media supplemented with 10% foetal bovine serum (FBS) and 1% antibiotics (GIBCO, USA). The cells were seeded in 24-well plates at a density of 50,000 cells per well and incubated in a CO₂ incubator at 37° C. After 24 hours, the media was removed and replaced with fresh media. The following day, the cells reached full confluence in each well of the 24-well plates and were subsequently employed for the migration assay. A straight scratch was created on the confluent cell monolayer using a sterile pipette tip, and then the cells were treated with 10 μg/ml of mitomycin C for 2 hours (Sigma, USA) to inhibit cell proliferation.

After the incubation period, the 24-well plates were removed from the incubator, and the media was replaced with fresh media supplemented with 100 μl of conditioned media (CM) collected from Native and powdered AM or 50 μl lyophilized and non-lyophilized exosomes (equivalent volume to 10 μg) or 100 μl CM obtained from a combination of Exosome/AM. Images of the scratch were captured at 0 hours and 24 hours to analyse wound closure. The percentage of closed area was calculated using ImageJ software, and the resulting data was plotted using Graph Prism software for further analysis and visualization.

b. Results

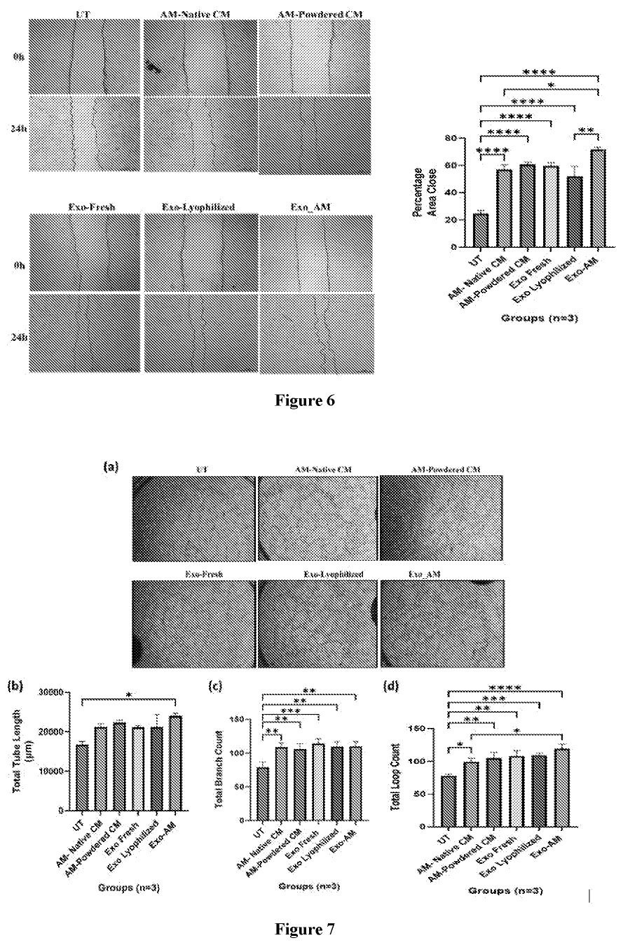

The functionality assay revealed that both the components of the final product formulation maintained their functionality in lyophilized form and were comparable to their native form. Moreover, they were able to significantly enhance cell migration with respect to untreated groups. Further, the combination of the freeze-dried AM and Exosome also showed significantly enhanced cell migration (FIG. 6).

### 3B. Angiogenesis Assay (Tube Formation Assay)

The tube formation assay is a vital tool for evaluating the wound healing potential of various products by assessing angiogenesis. This assay is valuable for assessing biomaterials and medical devices designed for wound healing applications, offering a predictive measure of in vivo efficacy. Its ability to replicate in vivo angiogenic processes makes it an indispensable tool for researchers and developers in the field of wound healing.

In a tube formation assay using Human Umbilical Vein Endothelial Cells (HUVECs), parameters such as tube length, loop count, and branch count are crucial for assessing angiogenesis and the formation of capillary-like structures. Each of these parameters have some significance as mentioned below:
a) Tube Length: It is a fundamental measure of angiogenesis and represents the overall extent of capillary-like structure formation. Longer tube lengths typically indicate a more robust angiogenic response. An increase in tube length suggests enhanced endothelial cell migration, proliferation, and organization, reflecting positive angiogenic activity. Conversely, a decrease may indicate an inhibitory effect on angiogenesis.
b) Loop Count: It is indicative of the complexity and maturation of the formed capillary network. Counting loops help evaluate the intricacy of the vascular structure. A higher loop count signifies a more mature and interconnected capillary network, reflecting a positive angiogenic response. Reduced loop count may indicate impaired vessel maturation or network complexity.
c) Branch Count: Branching points represent the formation of secondary vessels and branching events in the capillary-like network. Branch count is a measure of the vascular network's complexity. An increased branch count indicates a highly branched and interconnected vascular network, reflecting a positive angiogenic response. A decrease in branching points may suggest inhibited vessel sprouting and branching.
   a. Methodology

Human Umbilical Vein Endothelial Cells (HUVEC) were isolated from the Umbilical cord (obtained from donors undergoing cesarean surgery for delivery in AIIMS, New Delhi) using previously standardized protocols. The prior consent was taken from the donors and all serological parameters were evaluated as per DBT-ICMR guidelines (HIV, HBV, HCV test). These cells from different donors were pooled to avoid batch to batch variations. Geltrex (Thermo, USA), a matrix substitute for Matrigel, was used to coat the 15 well plate, creating a supportive environment for angiogenesis. Then 20,000 HUVECs were seeded onto the Geltrex-coated surface and treated with 100 μl of conditioned media (CM) collected from Native and powdered AM or 50 μl lyophilized and non-lyophilized exosomes (equivalent volume to 10 μg) or 100 μl CM obtained from a combination of Exosome/AM.

The plate was then incubated at 37° C. After an incubation period of 24 h, tube formation was assessed using an inverted phase-contrast microscope, capturing images for subsequent analysis. Parameters (FIG. 7a) such as total tube length, loop counts, and branch points were quantified using Tube Formation FastTrack AI Image Analysis software (Germany). Statistical analysis was performed to evaluate differences between experimental groups (FIG. 7b).

b. Results

The functionality assay revealed that both the components of the final product formulations maintained their functionality in lyophilized form and were comparable to their native form. Moreover, they were able to significantly induce a greater number of Loop and branches as well as total tube length. Further, the combination of the freeze-dried AM and Exosome also showed significantly better results (FIG. 7b).

### 3C. Total Collagen Content Assay

a. Methodology

Collagen content in the CM collected from native and powdered AM was assessed using Collagen Assay Kit (Cat No. MAK322, Sigma, USA) as per the manufacturer's instructions. Briefly, in the first step of this procedure, collagen in the sample was enzymatically digested into peptides. Subsequently, the N-terminal glycine containing peptides reacted with the dye reagent to form a fluorescent complex. The fluorescence intensity of this product, measured at λex/em=375/465 nm, was directly proportional to collagen concentration in the sample.

b. Results

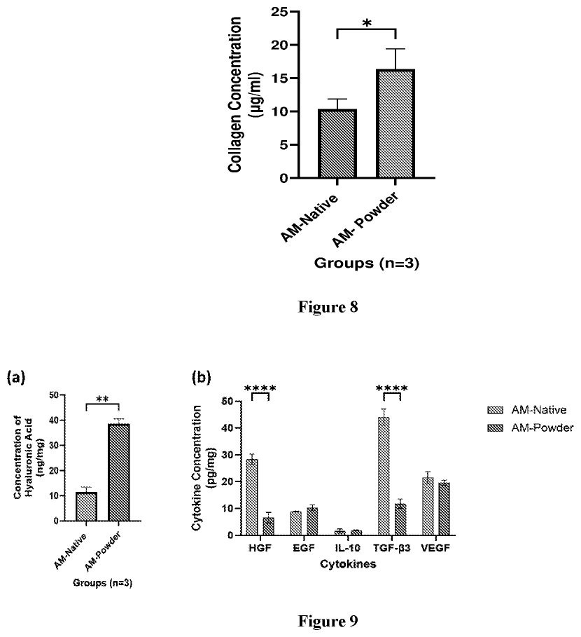

The total collagen content was observed to be significantly higher in Powder form of AM as compared to Native form (FIG. 8).

### 3D. Cytokine Assessment

a. Methodology

An ELISA was performed to measure the contents of the growth factors EGF, IL-10, HGF, Hyaluronic Acid (HA), TGF-β3 and VEGF (G-Biosciences, USA). The presence of these cytokines was measured in the conditioned medium that was collected after 24 hours of incubation in Serum Free media at 37° C.

b. Results

The relative level of cytokine factors EGF, IL-10, HGF, Hyaluronic Acid (HA), TGF-β3 and VEGF were found to be variable in both forms of AM. HGF and TGF-β3 concentration was significantly reduced in powdered form of AM while HA concentration was significantly higher in the powdered form of AM. Further, concentration of other cytokines was almost similar in either form of AM (FIG. 9).

### Example 4: Stability Assessment of Both the Components of the Final Formulation

For stability studies, both components of AM were stored in a defined ratio (100 μg exosome and 5 mg of AM) for up to 6 months at different storage conditions i.e., 4° C., −20° C. and −80° C. The functionality of the final formulation was evaluated based on:
a) Cell migration assay
b) Tube formation assay

Briefly, after the termination of the respective time points, the freeze-dried combination formulation was resuspended in 1 ml sterile 1×PBS and incubated at 37° C. for 24 h. The CM consisting of AM secretion and resuspended exosomes was aliquoted and used for further assays.

### Results:

The functionality of the final formulation was maintained at all the time points and temperatures. The cell migration assay analysis indicates that the cell migration potential was significantly higher in the final formulation irrespective of the time and storage temperature. However, −80° C. storage condition for both 1 month and 3 months' time point was much better as compared to other two storage temperatures.

Further, the tube formation assay analysis indicates similar results where, the loop count and branch count were significantly higher in all the groups as compared to untreated but within the group comparisons which showed that −80° C. storage condition was much better than 4° C. and −20° C. Similarly, the total tube length was significantly higher in treatment groups stored at −80° C.

Thus, these results indicate that the freeze-dried form of final product formulation can be stored for short duration at 4° C. or i.e., a normal refrigerator which is available with the clinicians in all hospitals and dispensaries, while for longer duration the temperature <−20° ° C. is preferred.

### Example 5: Preclinical Efficacy Studies

To assess the efficacy of the present disclosure, a rodent model featuring deep excision skin wounds was employed. This rat model closely mimics intricate human wounds, rendering findings applicable to human healing. It allows researchers to scrutinize diverse stages of tissue repair, including inflammation, granulation tissue formation, re-epithelialization, and tissue remodelling, unravelling insights into cellular and molecular mechanisms. This model is instrumental for investigating scar formation, encompassing factors like collagen deposition modulation and tissue remodelling. Understanding these dynamics contributes to interventions aimed at minimizing scar formation. Moreover, it enables the exploration of the inflammatory response, pivotal for wound healing, aiding in the development of anti-inflammatory strategies. The model also facilitates the study of angiogenesis and vascularization during wound healing, shedding light on interventions' effects on blood vessel formation for optimal nutrient supply. In essence, the rat deep excision skin wound model proves invaluable in comprehending various aspects of wound healing and tissue repair, with relevance to collagen dynamics, making it an essential tool in preclinical research for advancing the understanding and developing effective treatments.

a. Methodology

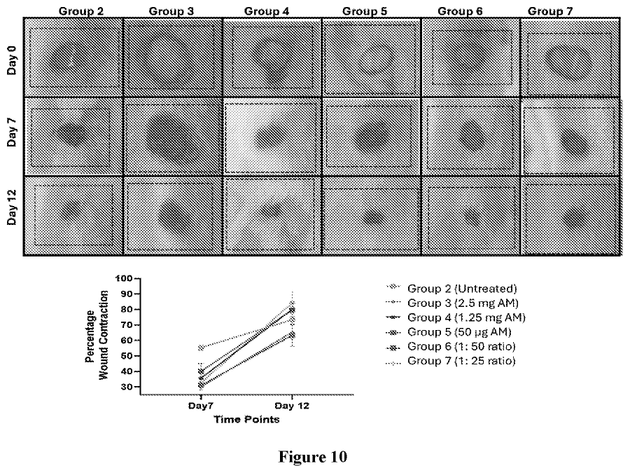

This study was conducted with 7 groups, each group (G2-G7) contained 5 animals and Group 1 served as healthy control (Table 1). Animals (Wistar rats) were obtained from the Central animal facility at AIIMS, New Delhi. The animals (G2-G7) were subjected to intraperitoneal anaesthesia with ketamine (80 mg/kg) and xylazine (10 mg/kg) to apply the wound excision procedure. The dorsal region of the animals was shaved and submitted to a lesion using 10 mm biopsy punch. Wounds were photographed, measured, and treated with topical formulations on Day 0. Size and percentage of wound contraction were measured at Day 7, and Day 12 (FIG. 10). Further, at Day 12 all rats were sacrificed, and wound tissue was processed for Histology sections preparations (Outsourcing).

TABLE 1
Number of groups and Dose study

| | | Dose | | Route of | Number of |
|---|---|---|---|---|---|
| Group | Treatment | per Animal | | Administration | Animal |
| G1 | Healthy | — | | | 5 |
| G2 | Negative Control | 100 μl 1x PBS | | Topical Application | 5 |
| G3 | AM Powder | 2.5 | mg | (Sprinkle over the | 5 |
| G4 | AM Powder | 1.25 | mg | wound at Day 0) | 5 |
| G5 | Exosome Powder | 50 | μg | | 5 |

| | | | |
|---|---|---|---|
| G6 | Combination Ratio A | 1:50 | 5 |
| G7 | Combination Ratio B | 1:25 | 5 |

b. Results

The Macroscopic Analysis was performed via Wound Contraction study. Through measurements made during the periods of 7, and 12 days, wound contraction percentages were analysed and reported. The wound contraction percentage was significantly higher for both the combinations as compared to untreated (FIG. 10).

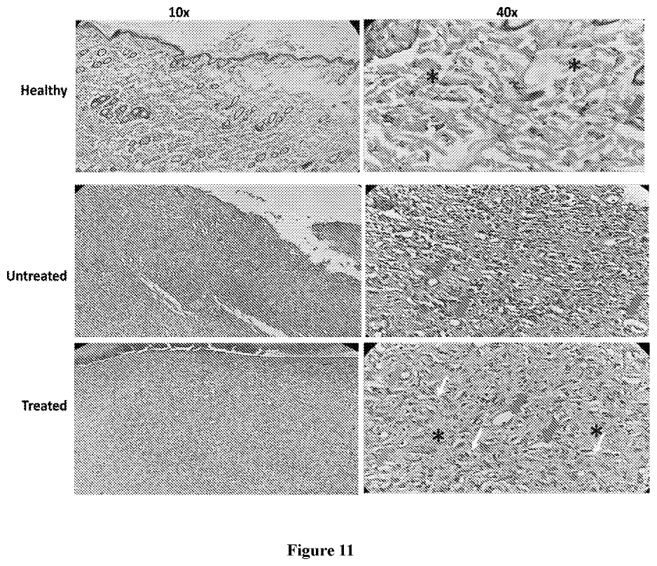

The microscopic examination focused on histological criteria, encompassing epithelization, granular tissue formation, inflammation, neo-vascularization, scab tissue development, and collagen formation and maturation (Table 2). A pathologist, presented with blinded slides to ensure an impartial evaluation, conducted the analysis. In comparison to the healthy group, the untreated group exhibited a noteworthy rise in inflammation, a lack of epithelization, limited vascular areas, and an absence of collagen maturation or rearrangement. However, the assessment and comparison of various treatments were based on the observed reversal and restoration of these parameters (FIG. 11).

The untreated groups (Group 2) showed severe inflammation both acute and chronic extending from the crust of wound till the subcutaneous fat tissue. Moreover, there was muscle cell disruptions observed in most of the untreated rat skin tissue. There was presence of scab tissue with high inflammatory cells and epithelization was absent. Neo-vascularization was observed, and the formation of granulation tissue was initiated. The collagen formation and orientation were not significant.

In contrast to untreated groups, both AM groups showed significant reduction of inflammation. There was presence of sab tissue and wound contraction was absent. Mostly proliferating fibroblast & capillaries were present along with collagenization. The healing phase appeared to be initiated.

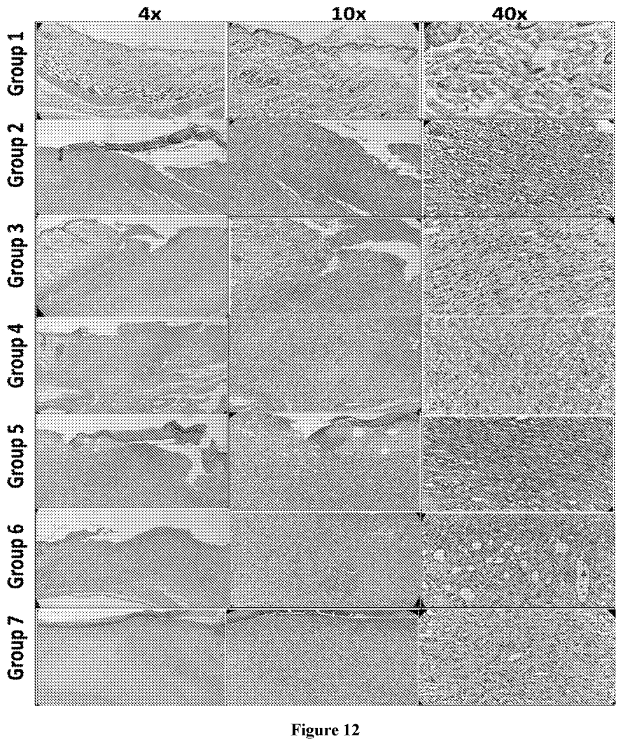

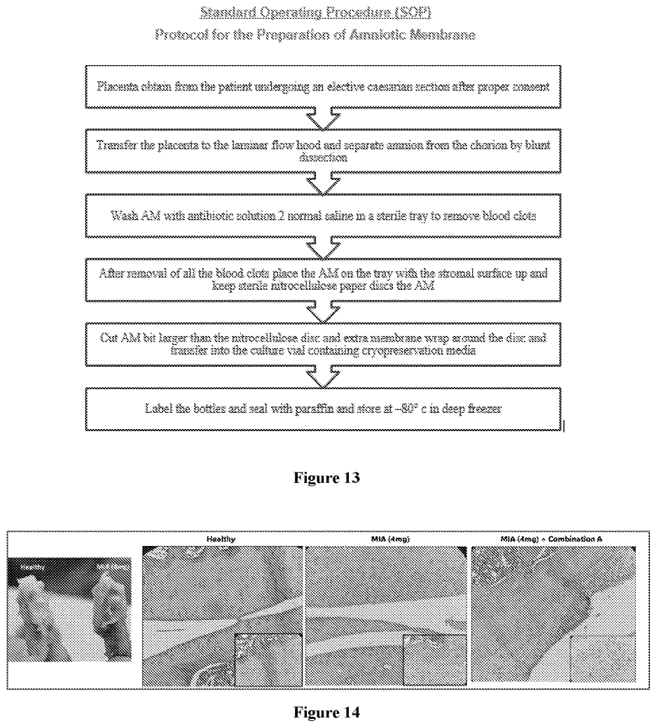

Further, the Exosome treated groups showed enhanced healing phase as compared to AM group where collagenization was present with mature collagen and fibroblast. The exudative area was observed to be reduced indicating reduced inflammation and enhanced granulation tissue formation. Also, the wound interface had low acute inflammation however, the epithelization was not there (FIG. 12).

TABLE 2
Histological Analysis of the different treatment groups

| | Group 2 | Group 3 | Group 4 | Group 5 | Group 6 | Group 7 |
|---|---|---|---|---|---|---|
| Parameters | Untreated | 2.5 mg AM | 1.25 mg AM | 50 μg Exo | 1:50 | 1:25 |
| Epithelization | ± | ± | ± | ± | ± | ± |
| Inflammation | +++ | + | + | + | ± | ± |
| Granulation | + | + | + | ++ | ++ | ++ |
| Tissue | | | | | | |
| Neo- | + | ++ | ++ | ++ | ++ | ++ |
| vascularization | | | | | | |
| Fibroblast | − | ± | ± | ± | ++ | ++ |
| Collagen | − | ± | ± | ++ | ++ | +++ |
| Rearrangement | | | | | | |
| Collagen | − | ± | ± | ++ | +++ | +++ |
| Maturation | | | | | | |

Overall, both the final combination ratio showed significant wound healing phase as indicated by neovascularization, mature granulation tissue, proliferating fibroblast, reduced inflammation, collagen formation and maturation.

### Example 6: Efficacy Study in Osteoarthritis Model

The AM powder as outlined in Example 1 and 2 was further studied for efficacy studies in the osteoarthritis (OA) model.

After obtaining ethical clearance from IAEC, 4-6 weeks old Wistar Rats, were procured and housed in Central Animal Facility, AIIMS in a 12-h light and dark cycle and grown in individual ventilated cages with access to food and water ad libitum. The rats were anesthetized by an intraperitoneal injection of ketamine (80 mg/kg body weight) and xylazine (12.5 mg/kg body weight). Osteoarthritis was induced through a single intra-articular injection of monosodium iodoacetate (MIA). Briefly, MIA (cat. #12512; Sigma, St. Louis, MO, where USA) was dissolved in 50 μl of sterile saline, and the dose of MIA was 4 mg. Under anaesthesia, the right knee was shaved and disinfected. Then, an incision was made at the centre of the knee to expose the patellar ligament.

Each rat was positioned on its back, and the leg was flexed 90° at the knee joint. The patellar ligament was palpate below the patella, and MIA was injected into the medial side of the ligament of right knee using a 29-gaugeneedle. Care was taken to ensure that the needle was not advanced too far into the cruciate ligaments. One week later after development of OA as evidenced by swelling of the injected knees, the rats were randomly divided into respective groups (Untreated/sham and Exosome based formulation-final ratio 1:50) and a single intra-articular injection of 50 μl saline, or exosome formulation or positive control was given. The rats were sacrificed at Day 28 for histological analysis.

### Results

The gross image analysis of the Rat knee indicated reduced swelling in treated group and no edema presence. Further hematoxylin and eosin (H&E) image analysis showed proliferation of spindle shaped cells and multilayered structure in treated group as compared to untreated/sham. There was no inflammation observed after injecting the product formulation indicating that there was no immune response initiated due to the product (FIG. 14).

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

It is to be noted that the present disclosure is susceptible to modifications, changes, and adaptations by those skilled in the art. Such modifications, changes adaptations are intended to be within the scope of the present disclosure.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

### Advantages

(1) The composition shows enhanced therapeutic effect due to the regenerative effect of the exosomes derived from wharton's jelly stem cells and helps in treating by targeting the damaged cells and tissues.
(2) Use of exosomes instead of stem cells alleviate the limitations of the cell-based approach.
(3) The novel composition contains amniotic membrane that serves as a substrate for regrowth of deficient epithelium for promoting the angiogenesis, its natural anti-inflammatory and anti-scarring properties, secretory growth factors such as VEGF, ANG1, TIMP-1, IL-1B, PDGF, bFGF, HGF. It also consists of collagen, hyaluronic acid, fibronectin laminin, etc that help in building up the tissue matrix.
(4) The raw material used in the present formulation is a biological waste, therefore manufacturing cost of the product can be reduced.
(5) Suitable for allogenic applications due to the absence of HLA-Class II molecules on stem cell derived exosomes and using the denudation process for amniotic membrane preparation.

The claims:

1. A composition comprising exosomes and amniotic membrane,
wherein the exosome and amniotic membrane are present in a therapeutically effective ratio range of 1:10 to 1:50.

2. The composition as claimed in claim 1, wherein the exosomes are derived from wharton's jelly stem cells.

3. The composition as claimed in claim 1, wherein the amniotic membrane is derived from placental tissue.

4. The composition as claimed in claim 1, wherein the exosome and amniotic membrane are freeze dried and are in a powdered form.

5. The composition as claimed in claim 1, wherein the exosome powder and amniotic membrane powder is preferably present in a ratio of about 1:25:1:50.

6. The composition as claimed in claim 1, wherein the composition is a freeze-dried nano-formulation.

7. The composition as claimed in claim 1, wherein the composition is in the form of a powder, spray, gel, ointment, liquid.

8. The composition as claimed in claim 1, wherein the composition can be administered topically, intraarticularly, intravenously, subcutaneously at the site of injury.

9. A method of preparing a composition as claimed in claim 1 comprising the steps of:
a) Lyophilizing exosomes at −50° C. for 24 hrs to obtain exosome powder;
b) optionally solubilizing amniotic membrane and then lyophilizing at −50° C. for 24 hrs to obtain amniotic membrane powder;
c) sterilizing the powders obtained in step a) and b); and
d) mixing the sterilized exosome and amniotic membrane powder obtained in step c) in a ratio range of 1:10 to 1:50 to obtain the final composition.

10. The method as claimed in claim 1, wherein the amniotic membrane is solubilized by an enzymatic method.

11. A kit comprising the composition as claimed in claim 1 along with an instruction manual.

10. The composition as claimed in claim 1, as and when used for healing wounds or treatment of bone degenerative diseases selected from but not limited to osteoarthritis, osteoporosis, osteopenia, osteonecrosis.

11. The composition as claimed in claim 1, wherein the composition is in the form of a powder, gel, spray when used for wound healing.

12. The composition as claimed in claim 1, wherein the composition is in the form of a powder which can be later solubilized to be used as an intra-articular injection for treatment of bone-cartilage degenerative disorders.

13. A method of treating wound by applying the composition as claimed in claim 1.

14. A method of treating bone-cartilage degenerative diseases by administering the composition as claimed in claim 1 intraarticularly, intravenously, subcutaneously or at the site of injury.
